(19) United States
(12) Patent Application Publication
(10) Pub. No.: US 2026/0076681 A1
(43) Pub. Date: Mar. 19, 2026

(54) LEFT ATRIAL APPENDAGE CLOSURE DEVICE WITH MECHANICAL JOINING ASSEMBLY

(71) Applicant: BOSTON SCIENTIFIC SCIMED, INC., Maple Grove, MN (US)

(72) Inventors: Neal Eidenschink, Wayzata, MN (US); Nicholas Turowski, Maple Grove, MN (US)

(73) Assignee: BOSTON SCIENTIFIC SCIMED, INC., MAPLE GROVE, MN (US)

(21) Appl. No.: 19/330,077

(22) Filed: Sep. 16, 2025

Related U.S. Application Data
(60) Provisional application No. 63/695,478, filed on Sep. 17, 2024.

(51) Int. Cl. A61B 17/12; A61B 17/00

(52) U.S. Cl. CPC A61B 17/12122; A61B 17/12172; A61B 2017/00477

(57) ABSTRACT

A left atrial appendage closure device includes an expandable frame that extends between a proximal region and a distal region, with the distal region including a plurality of tines each having an engagement portion. A connection assembly secures the engagement portion of each of the plurality of tines together. The connection assembly includes a connection member having an outer surface and a ring member that is advanced over the outer surface of the connection member, the ring member including an inner annular surface and a plurality of engagement recesses extending from the inner annular surface, each of the plurality of engagement recesses accommodating the engagement portion of a corresponding one of the plurality of tines. The engagement portion of each of the plurality of tines are entrapped between the ring member and the connection member, thereby securing the plurality of distal tines.

### CROSS REFERENCE TO RELATED APPLICATIONS

This application claims the benefit of priority of U.S. Provisional Application No. 63/695,478 filed September 17, 2024, the entire disclosure of which is hereby incorporated by reference.

### TECHNICAL FIELD

The disclosure relates generally to medical devices and more particularly to left atrial appendage closure devices.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device. The LAAC device includes an expandable frame that extends between a proximal region and a distal region including a plurality of tines each having an engagement portion. A connection assembly is adapted to secure together the engagement portion of each of the plurality of tines. The connection assembly includes a connection member having an outer surface and a ring member that is adapted to be advanced over the outer surface of the connection member. The ring member includes an inner annular surface and a plurality of engagement recesses that extend from the inner annular surface. Each of the plurality of engagement recesses are adapted to accommodate the engagement portion of a corresponding one of the plurality of tines. The engagement portion of each of the plurality of tines is entrapped between the ring member and the connection member, thereby securing the plurality of distal tines.

Alternatively or additionally, the ring member and the connection member may be adapted to provide a threaded engagement therebetween.

Alternatively or additionally, the ring member and the connection member may be adapted to provide a mechanical engagement therebetween.

Alternatively or additionally, the ring member and the connection member may be adapted to provide a snap fit engagement therebetween.

Alternatively or additionally, the engagement portion of each of the plurality of tines extending between the ring member and the connection member may be at least substantially parallel with a longitudinal axis extending through the connection member.

Alternatively or additionally, a portion of each of the plurality of tines outside of the ring member may be at least substantially orthogonal to the longitudinal axis extending through the connection member.

Alternatively or additionally, the ring member may be adapted to be advanced over the connection member after the securement portion of each of the plurality of distal tines are disposed within a corresponding one of the plurality of engagement recesses defined within the inner annular surface of the ring member.

Alternatively or additionally, the connection member may include a tapered surface in order to allow the ring member to be advanced over the connection member.

Alternatively or additionally, the connection member may include a distal plate that limits relative distal travel of the plurality of distal tines.

Alternatively or additionally, the connection member may include one or more axially extending slots that facilitate advancing the ring member over the connection member.

Another example may be found in a left atrial appendage closure (LAAC) device. The LAAC device includes an expandable frame that extends between a proximal region and a distal region including a plurality of tines each having an engagement portion, a connection member having an outer surface, and a ring member that is adapted to be advanced over the outer surface of the connection member in order to entrap the engagement portion of each of the plurality of tines between the ring member and the connection member. The ring member includes a plurality of engagement recesses that are adapted to accommodate the engagement portion of a corresponding one of the plurality of tines.

Alternatively or additionally, the plurality of engagement regions may be arranged in order to equally space the engagement portions of each of the plurality of tines.

Alternatively or additionally, the connection member may include an annular body portion.

Alternatively or additionally, the annular body portion may include a reduced diameter segment that is adapted to accommodate the ring member therein.

Alternatively or additionally, the connection member may include a distal segment including a surface that limits distal travel of the plurality of distal tines.

Alternatively or additionally, the distal segment may include an atraumatic distal segment.

Alternatively or additionally, the engagement portion of each of the plurality of tines may be adapted to engage a proximal surface of the ring member in order to limit axial travel of the engagement portion relative to the ring member.

Alternatively or additionally, the LAAC device may further include an occlusive member that extends over at least part of the proximal region of the expandable frame.

Another example may be found in a left atrial appendage closure (LAAC) device. The LAAC device includes an expandable frame that extends between a proximal region and a distal region including a plurality of tines each having an engagement portion, a connection member, and a ring member that is adapted to engage the engagement portion of each of the distal tines prior to advancing the ring member over the connection member to secure the ring member in place relative to the connection member.

Alternatively or additionally, advancing the ring member over the connection member may serve to secure the engagement portion of each of the distal tines between the ring member and the connection member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative LAAC device;

FIG. 2 is a distal end view of an illustrative expandable frame forming part of the illustrative LAAC device of FIG. 1;

FIG. 3 is an enlarged view of a portion of the illustrative expandable frame of FIG. 2;

FIG. 4 is an end view showing a portion of the illustrative expandable frame of FIG. 2 in combination with an illustrative connection assembly;

FIG. 5 is a perspective view of the illustrative connection assembly of FIG. 4;

FIG. 6 is a side view of an illustrative connection member usable in the illustrative connection assembly of FIG. 5;

FIG. 7 is an end view of an illustrative connection member usable in the illustrative connection assembly of FIG. 5;

FIG. 8 is a side view of an illustrative connection member usable in the illustrative connection assembly of FIG. 5;

FIG. 9 is a side view of an illustrative connection member usable in the illustrative connection assembly of FIG. 5;

FIG. 10 is a first perspective view of a portion of the illustrative expandable frame of FIG. 2 in combination with an illustrative connection assembly;

FIG. 11 is a second perspective view of a portion of the illustrative expandable frame of FIG. 2 in combination with the illustrative connection assembly of FIG. 10;

FIG. 12 is a perspective view of a portion of the illustrative expandable frame of FIG. 2 in combination with an illustrative connection assembly that is welded together; and

FIG. 13 is a schematic view of a number of possible shapes for the engagement portions of the distal tines forming part of the illustrative expandable frame of FIG. 2.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified .

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A left atrial appendage closure (LAAC) device may be used to close off or otherwise occlude the volume within a left atrial appendage from the rest of the left atrium in a mammalian heart. Preventing blood flow in and out of the left atrial appendage can reduce the possibility of embolisms from forming within the left atrial appendage and reaching other parts of the body, including the brain. In some instances, a left atrial appendage closure (LAAC) device includes an expandable frame that extends between a proximal region and a distal region having a plurality of tines each having an engagement portion. The LAAC device includes a connection assembly that is adapted to secure together the engagement portion of each of the plurality of tines. The connection assembly includes a connection member having an outer surface and a ring member that is adapted to be advanced over the outer surface of the connection member. The ring member includes an inner annular surface and a plurality of engagement recesses that extend from the inner annular surface and that are each adapted to accommodate the engagement portion of a corresponding one of the plurality of tines. The engagement portion of each of the plurality of tines is entrapped between the ring member and the connection member, thereby securing the plurality of distal tines.

In some cases, the ring member and the connection member may be adapted to provide a threaded engagement therebetween. In some cases, the ring member and the connection member may be adapted to provide a mechanical engagement therebetween. In some cases, the ring member and the connection member may be adapted to provide a snap fit engagement therebetween.

In some cases, the engagement portion of each of the plurality of tines extending between the ring member and the connection member may be at least substantially parallel with a longitudinal axis extending through the connection member. Substantially parallel may be defined as the engagement portion of each of the plurality of tines extending between the ring member and the connection member being within twenty percent or less of parallel with the longitudinal axis, or being within ten percent or less of parallel with the longitudinal axis. A portion of each of the plurality of tines outside of the ring member may be at least substantially orthogonal to the longitudinal axis extending through the connection member. Substantially orthogonal may be defined as a portion of each of the plurality of tines outside of the ring member being within twenty percent or less of orthogonal to the longitudinal axis extending through the connection member, or being within ten percent or less of orthogonal to the longitudinal axis extending through the connection member.

In some cases, the ring member may be adapted to be advanced over the connection member after the securement portion of each of the plurality of distal tines are disposed within a corresponding one of the plurality of engagement recesses defined within the inner annular surface of the ring member. The connection member may be adapted to allow the ring member to be advanced over the connection member. In some cases, the connection member may include a tapered surface. In some cases, the connection member may include one or more axially extending slots that facilitate advancing the ring member over the connection member.

In some instances, a left atrial appendage closure (LAAC) device includes an expandable frame extending between a proximal region and a distal region having a plurality of tines each having an engagement portion. A connection member has an outer surface and a ring member is adapted to be advanced over the outer surface of the connection member in order to entrap the engagement portion of each of the plurality of tines between the ring member and the connection member. The ring member includes a plurality of engagement recesses that are adapted to accommodate the engagement portion of a corresponding one of the plurality of tines.

In some cases, the plurality of engagement regions may be arranged in order to equally space the engagement portions of each of the plurality of tines. In some cases, the connection member may include an annular body portion. In some cases, the annular body portion may include a reduced diameter segment that is adapted to accommodate the ring member therein. In some cases, the connection member may include a distal segment including a surface that limits distal travel of the plurality of distal tines. In some cases, the distal segment may include an atraumatic distal segment. In some cases, the engagement portion of each of the plurality of tines may be adapted to engage a proximal surface of the ring member in order to limit axial travel of the engagement portion relative to the ring member. The LAAC device may further include an occlusive member that extends over at least part of the proximal region of the expandable frame.

In some instances, a left atrial appendage closure (LAAC) device includes an expandable frame extending between a proximal region and a distal region. The distal region includes a plurality of tines each having an engagement portion. The LAAC device includes a connection member and a ring member that is adapted to engage the engagement portion of each of the distal tines prior to advancing the ring member over the connection member to secure the ring member in place relative to the connection member. In some cases, advancing the ring member over the connection member may serve to secure the engagement portion of each of the distal tines between the ring member and the connection member.

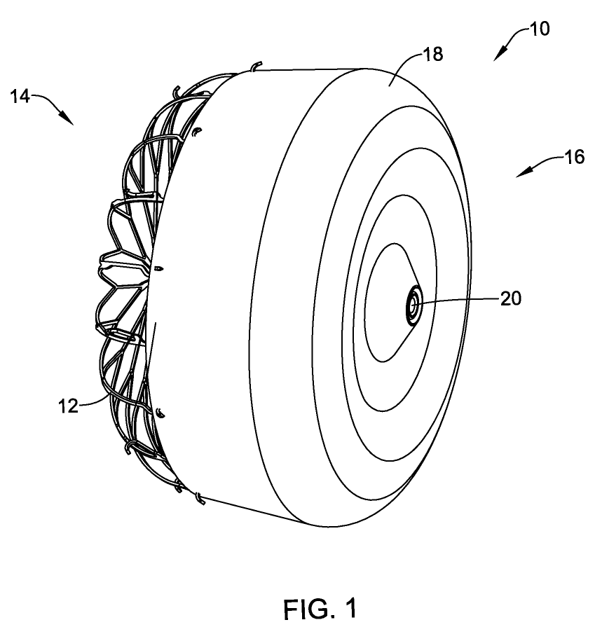

FIG. 1 is a side view of an illustrative LAAC device 10 that may be adapted to be implanted within a patient’s LAA in order to occlude the patient’s LAA and thus reduce or even eliminate the possibility for a blood clot within the LAA to exit the LAA and enter the patient’s vasculature. An exemplary LAAC device 10 includes WATCHMAN FLX™ and WATCHMAN FLX™ Pro available from Boston Scientific.

The LAAC device 10 may include an expandable frame 12 that may be considered as including a distal region 14 and a proximal region 16. The expandable frame 12 may be movable between a collapsed configuration for delivery and an expanded configuration (as shown) when deployed. The LAAC device 10 may be adapted to be implanted within a patient’s left atrial appendage in order to reduce or even eliminate blood flow into or out of the left atrial appendage. In some cases, an occlusive member 18 extends over at least a portion of the proximal region 16. The expandable frame 12 may define a hub 20 that allows for a delivery device (not shown) to be releasably secured to the LAAC device 10 for delivery and deployment. The hub 20 is adapted to allow a delivery device to be disconnected after the LAAC device 10 has been deployed, allowing the LAAC device 10 to remain behind in position within the patient’s left atrial appendage.

In some cases, the occlusive member 18 may be permeable or impermeable to blood and/or other fluids, such as water. The occlusive member 18 may include a woven fabric/material or mesh, a non-woven fabric/material or mesh, a braided and/or knitted material, a fiber, a sheet-like material, a fabric, a mesh, a fabric mesh, a polymeric membrane, a metallic or polymeric mesh, a porous filter-like material, a covering, and/or other suitable construction, for example. In some cases, the occlusive member 18 may prevent thrombi (i.e. blood clots, etc.) from passing through the occlusive member 18 and out of the LAA into the blood stream. In some cases, the occlusive member 18 may promote endothelialization after implantation, thereby effectively removing the left atrial appendage from the patient’s circulatory system.

The occlusive member 18 may be formed from a suitable material such as polyethylene terephthalate, polyester, nylon, acrylic materials, a polyolefin, and/or the like, combinations thereof, and/or other materials disclosed herein. In other instances, the occlusive material may include a metallic mesh formed from nickel-titanium alloy, stainless steel, titanium, other materials disclosed herein, combinations thereof, and/or the like.

In some examples, the expandable frame 12 may be integrally formed and/or cut from a unitary member. In some cases, the expandable frame 12 and may be integrally formed and/or cut from a unitary tubular member and subsequently formed and/or heat set to a desired shape in the expanded configuration. In some cases, the expandable frame 12 may be integrally formed and/or cut from a unitary flat member, and then rolled or formed into a tubular structure and subsequently formed and/or heat set to the desired shape in the expanded configuration. Some exemplary means and/or methods of making and/or forming the expandable frame 12 include laser cutting, machining, punching, stamping, electro discharge machining (EDM), chemical dissolution, etc. Other means and/or methods are also contemplated.

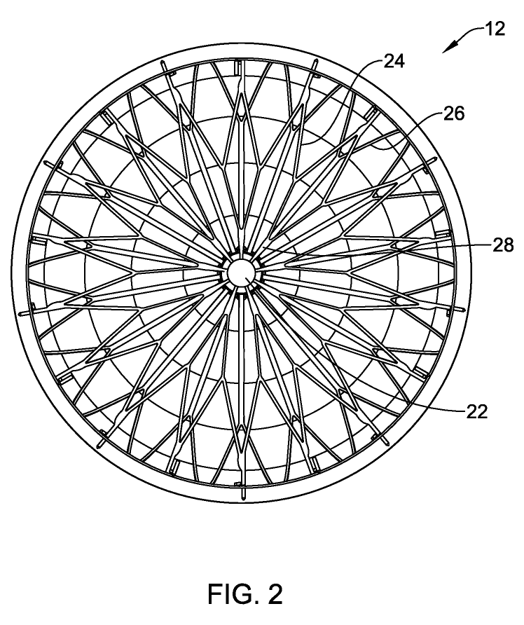

The expandable frame 12 may include a number of frame elements. As an example, the expandable frame 12 includes a number of frame elements 24 (only one is labeled in FIG. 2) that together form the distal region 14 of the expandable frame 12. The expandable frame 12 includes a number of frame elements 26 (only one is labeled in FIG. 2) that together form the proximal region 16 of the expandable frame 12. The frame elements 24, each of which may include one, two, three or more individual segments, terminate together in a distal tine 28 (only one is labeled in FIG. 2). In some cases, each distal tine 28 may bifurcate into two individual segments, or may “tri-burcate” into three individual segments. Similarly, the frame elements 26, each of which may include one, two, three or more individual segments, terminate together at or near the hub 20. The expandable frame 12 may include any number of frame elements 24 and any number of frame elements 26. In some cases, each of the frame elements 24 may be considered as extending distally from a corresponding one of the frame elements 26. In some cases, each of the frame elements 26 may be considered as extending proximally from a corresponding one of the frame elements 24.

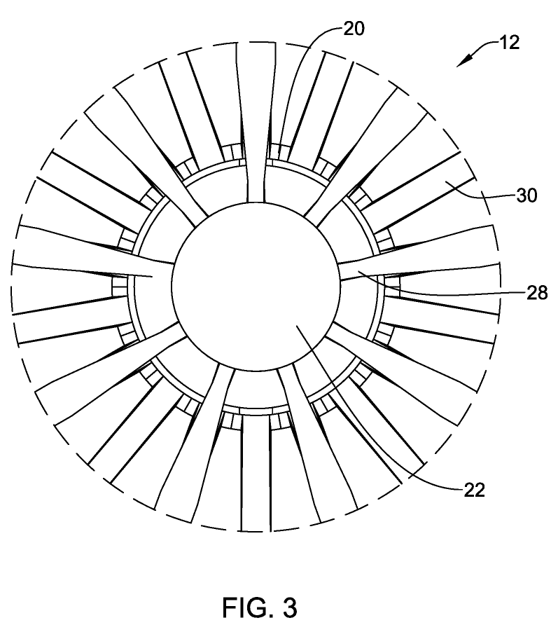

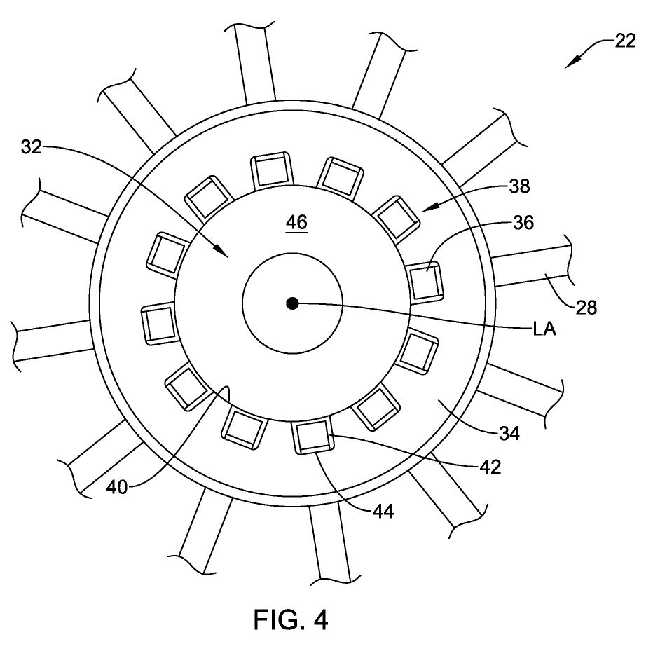

FIG. 2 is a distal end view of the expandable frame 12 and FIG. 3 is an enlarged distal end view of a central portion of the expandable frame 12. A connection assembly 22 may be seen in the middle of the distal end view, and will be described in greater detail with respect to FIGS. 4, 5 and 6. In some cases, the ends of the distal tines 28 may be joined together via the connection assembly 22. In some cases, the connection assembly 22 may enable the ends of the distal tines 28 to be joined together in a manner that does not require welding the ends of the distal tines 28 to any other structure (including the connection assembly 22). In some cases, the distal connection 22 may enable a mechanical fixation of the ends of the distal tines 28. Also shown in FIG. 3 are the proximal struts 30 that serve to terminate the frame elements 26 and that are fixed to the hub 20.

It will be appreciated that the number of distal tines 28 may vary, depending on the exact design and size of the expandable frame 12. FIGS. 2 and 3 show an expandable frame 12 that includes a total of nine distal tines 28. In some cases, the expandable frame 12 may have fewer than nine distal tines 28. For example, the expandable frame 12 may have six, seven or eight distal tines 28. In some cases, the expandable frame 12 may have more than nine distal tines 28. For example, the expandable frame 12 may have ten, eleven, twelve or more distal tines 28. In some cases, the connection assembly 22 may be designed to accommodate any number of distal tines 28, depending on the overall design and construction of the expandable frame 12.

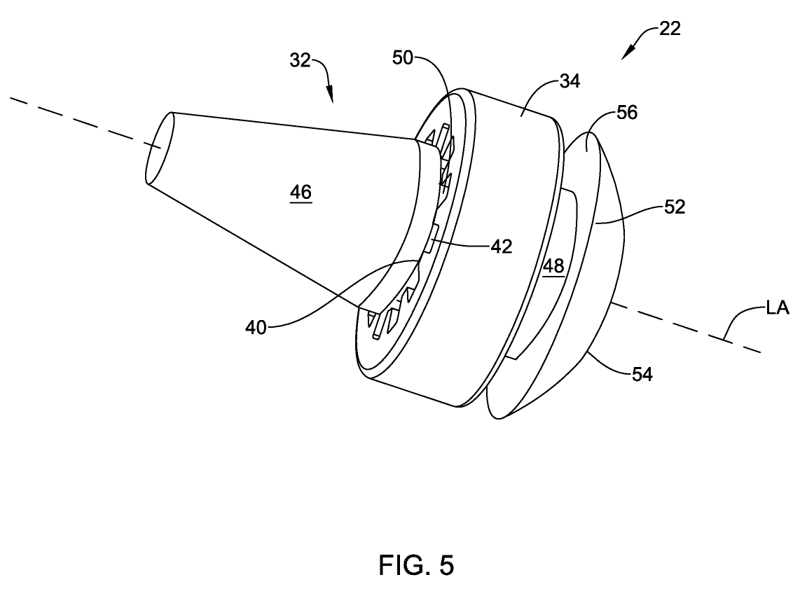

FIG. 4 is an end view of the illustrative connection assembly 22 shown in combination with the distal tines 28. As will be shown in greater detail in FIG. 5, the connection assembly 22 includes a connection member 32 and a ring member 34 that is coupled with the connection member 32. In some cases, the distal tines 28 (only one of which are labeled in FIG. 4) are coplanar or at least substantially coplanar with each other as the distal tines 28 approach the connection assembly 22, and then bend just before each distal tine 28 contacts the connection assembly 22. Substantially coplanar may be defined as each of the distal tines 28 being within twenty percent or less of being coplanar, or being within ten percent or less, for example.

While the bends are not visible in this view, it will be appreciated that the distal tines 28 are shown flat as the distal tines 28 approach the connection assembly 22 and an end 36 (only one of which is labeled in FIG. 4) of each distal tine 28 is visible extending through the ring member 34. In some cases, the portion of the distal tine 28 from where the distal tine 28 bends to the end 36 of each distal tine 28 may be considered as being an engagement portion 38 (only one of which are labeled in FIG. 4) of the distal tine 28. In some cases, each distal tine 28 may bend such that the engagement portion 38 is orthogonal to the rest of the distal tine 28. In some cases, the distal tine 28 may bend through a smaller angle.

The ring member 34 includes an inner annular surface 40 that is adapted to contact the connection member 32. In some cases, the inner annular surface 40 includes a number of tine engagement recesses 42 (only one of which are labeled in FIG. 4) that accommodates the end 36 of each distal tine 28 extending up through the corresponding engagement recess 42. In some cases, the plurality of engagement recesses 42 may be circumferentially equally spaced about the inner annular surface 40 such that the engagement portion 38 of each of the distal tines 28 are equally spaced.

The distal tine 28, or at least the engagement portion 38 of the distal tine 28, may be constrained from moving in a radially outward direction by virtue of the engagement portion 38 extending through the engagement recess 42, and contacting a radially outermost surface 44 of each of the engagement recesses 42. The distal tine 28, or at least the engagement portion 38 of the distal tine 28, may be constrained from moving in a radially inward direction because any radially inward movement of the engagement portion 38 of the distal tine 28 would result in the engagement portion 38 of the distal tine 28 contacting the connection member 32. The engagement portion 38 of each distal tine 28 may be thought of as being entrapped between the connection member 32 and the ring member 34. In some cases, the connection member 32 may include a tapered portion 46 (more visible in FIG. 5) that can help facilitate advancing the ring member 34 over the connection member 32. The tapered portion 46 may extend at any desired angle in order to allow advancement of the ring member 34 over the connection member 32. In some cases, the engagement portion 38 of each distal tine 28 may be disposed within the corresponding engagement recess 42 before advancing the ring member 34 over the connection member 32.

In some cases, the connection assembly 22 may be considered as including a longitudinal axis LA that extends through the connection assembly 22. In some cases, the engagement portion 38 of each of the plurality of distal tines 28 extending between the ring member 34 (and through a corresponding engagement recess 42) and the connection member 32 may be considered as being at least substantially parallel with the longitudinal axis LA. Substantially parallel may be defined as the engagement portion 38 and the longitudinal axis LA being within twenty percent or less of parallel, or within ten percent or less of parallel. In some cases, a portion of each of the distal tines 28 outside of the ring member 34 may be considered as being at least substantially orthogonal to the longitudinal axis LA. Substantially orthogonal may be defined as the engagement portion 38 and the longitudinal axis LA being within twenty percent or less of orthogonal, or within ten percent or less of orthogonal.

FIG. 5 is a perspective view of the connection assembly 22, showing additional details regarding the connection member 32 and the ring member 34. The connection member 32 includes an annular body portion 48. In some cases, the annular body portion 48 may include additional features to help secure the ring member 34 in place relative to the connection member 32. In some cases, as shown, the annular body portion 48 may include a reduced diameter segment 50 that has an outer diameter that is less than that of the rest of the annular body portion 48 (such as the visible portion of the annular body portion 48). In some cases, the reduced diameter segment 50 may have an outer diameter that is equal to or less than an inner diameter defined by the annular inner surface 40 of the ring member 34 while the rest of the annular body portion 48 has an outer diameter that is greater than an inner diameter defined by the annular inner surface 40.

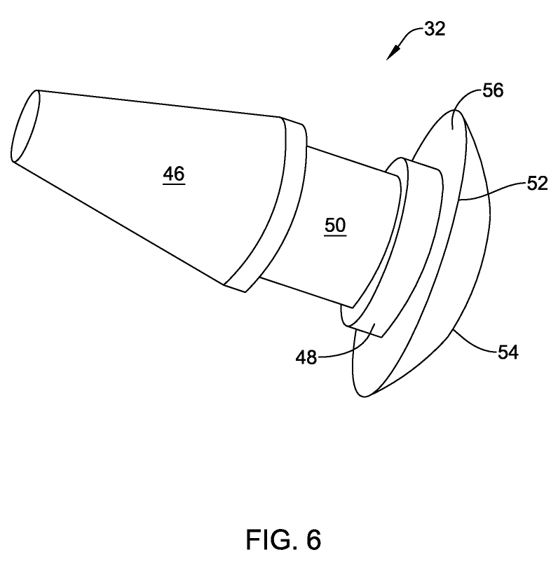

FIG. 6 shows how the reduced diameter segment 50 has an outer diameter that is less than that of the rest of the annular body portion 48. The connection member 32 may be considered as being adapted to provide a snap-fit coupling with the ring member 34. The connection member 32 includes a distal plate 52 having a distal surface 54 that will form a distal-most part of the LAAC device 10 once assembled. In some cases, the distal surface 54 may be adapted to be atraumatic, particularly when the LAAC device 10 is in a collapsed configuration for delivery. In some cases, the distal surface 54 may be curved. The distal plate 52 also includes a proximal surface 56 that can help limit axial travel of the engagement portion 38 of the distal tines 28. In some cases, inclusion of the distal plate 52 can help to provide a more uniform arrangement of the distal tines 28.

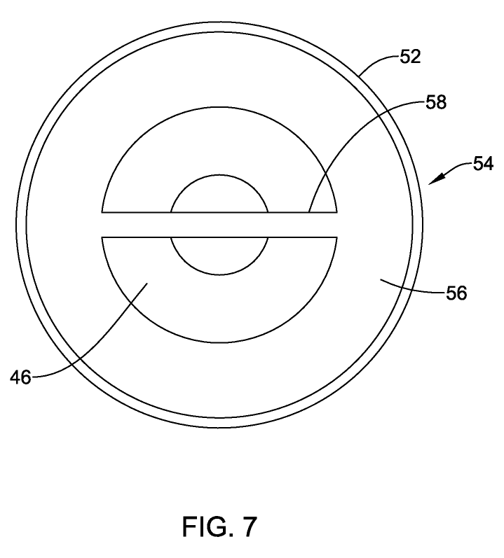

FIG. 7 is an end view of the illustrative connection member 32. In some cases, the connection member 32 may include a slot 58 that is cut or otherwise formed within the connection member 32. In some cases, the slot 58 may extend through the tapered portion 46. In some cases, the slot 58 may extend at least partially through the annular body portion 48. The slot 58 may allow the connection member 32 to temporarily reduce in diameter to facilitate advancement of the ring member 34 over the connection member 32. While a single slot 58 is shown, in some cases the connection member 32 may have two or more slots 58 that extend at least partially through the connection member 32. If the connection member 32 has two slots 58, the two slots 58 may be arranged orthogonally to each other (and still parallel with the longitudinal axis LA). If the connection member 32 has three slots 58, the three slots 58 may be arranged such that each of the slots 58 are parallel with the longitudinal axis LA and are each arranged about 120 degrees apart circumferentially. In some instances, the connection member 32 and the ring member 34 may be adapted to provide a mechanical engagement therebetween. As an example, the ring member 34 may be advanced over the connection member 32 only as far as the slot 58 allows the ring member 34 to be advanced.

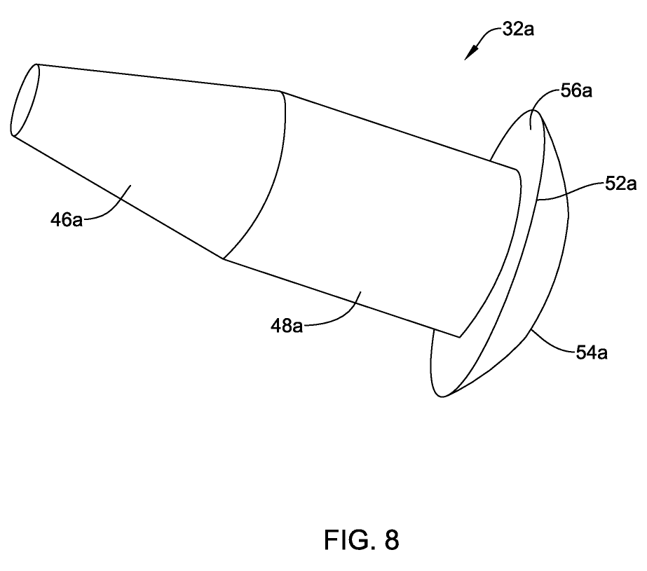

FIG. 8 is a side view of an illustrative connection member 32a that includes a tapered portion 46a, an annular body portion 48a that has a constant diameter (no reduced diameter segment) and a distal plate 52a having a distal surface 54a and a proximal surface 56a. Assuming that the inner annular surface 40 of the ring member 34 has a constant inner diameter, the ring member 34 may be physically pushed over the tapered portion 46a and over the annular body portion 48a until frictional forces prevent further advancement of the ring member 34.

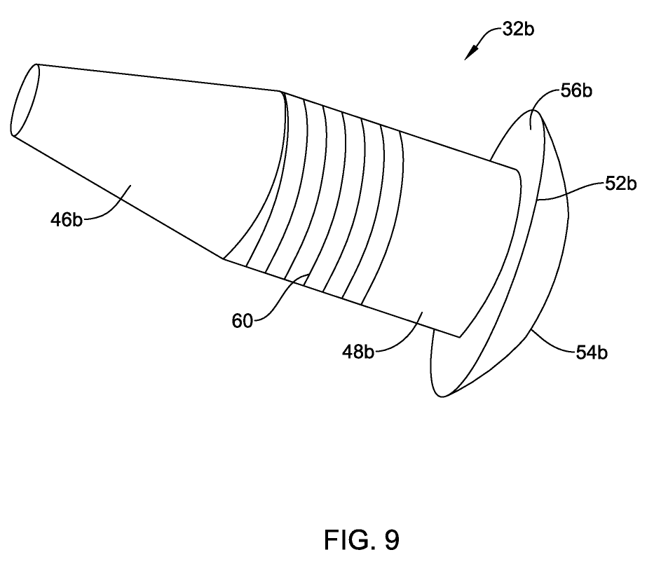

FIG. 9 is a side view of an illustrative connection member 32b that includes a tapered portion 46b, an annular body portion 48b that has a constant diameter (no reduced diameter segment) and a distal plate 52b having a distal surface 54b and a proximal surface 56b. A portion of the annular body portion 48b includes a threaded surface 60. In some cases, the inner annular surface 40 of the ring member 34 may include a corresponding threaded surface (not shown) that is adapted to threadedly engage the threaded surface 60. After the engagement portion 38 of each of the distal tines 28 has been engaged within a corresponding engagement recess 42, the ring member 34 may be advanced over the connection member 32b until the threaded surface of the ring member 34 engages the threaded surface 60. The ring member 34 may be rotated relative to the connection member 32b, thereby causing the ring member 34 to be advanced over the connection member 32b. The ring member 34 will travel as far as the threaded surface 60 allows.

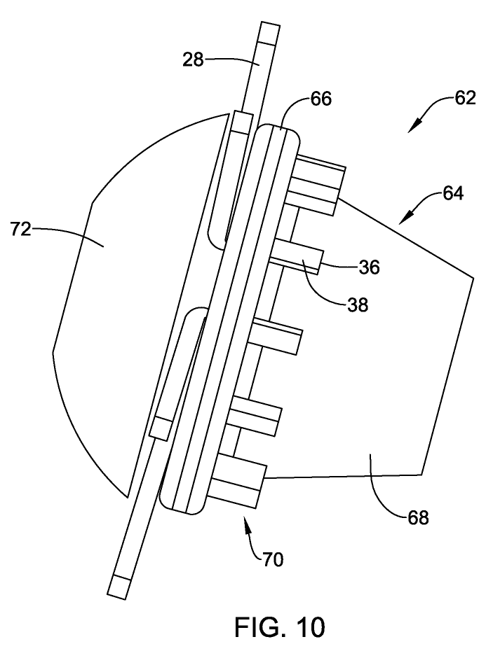

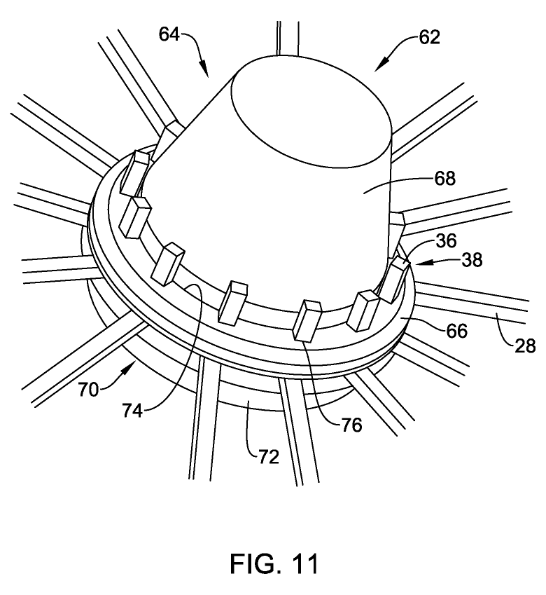

FIG. 10 is a first perspective view and FIG. 11 is a second perspective view of an illustrative connection assembly 62 shown engaged with the distal tines 28 of the expandable frame 12. The connection assembly 62 includes a connection member 64 and a ring member 66 that is adapted to advance over the connection member 64. The connection member 64 includes a tapered portion 68, an annular body portion 70 and an atraumatic distal section 72. The atraumatic distal section 72 will form the distal-most part of the expandable frame 12 and will help to prevent possible tissue damage as the LAAC device 10 is advanced through the vasculature in order to reach the left atrial appendage.

The ring member 66 includes an inner annular surface 74 that defines a plurality of engagement regions 76 (only one of which are labeled in FIG. 11). Each engagement region 76 accommodates one of the engagement portions 38 of one of the distal tines 28. In some cases, the connection member 64 and the ring member 66 may be adapted to provide a mechanical engagement therebetween. In some cases, the connection member 64 and the ring member 66 may be adapted to provide a swaged engagement therebetween. In some cases, the connection member 64 and the ring member 66 may be adapted to provide a snap-fit engagement therebetween (as shown for example in FIGS. 5 and 6). In some cases, the connection member 64 and the ring member 66 may be adapted to provide a threaded engagement therebetween.

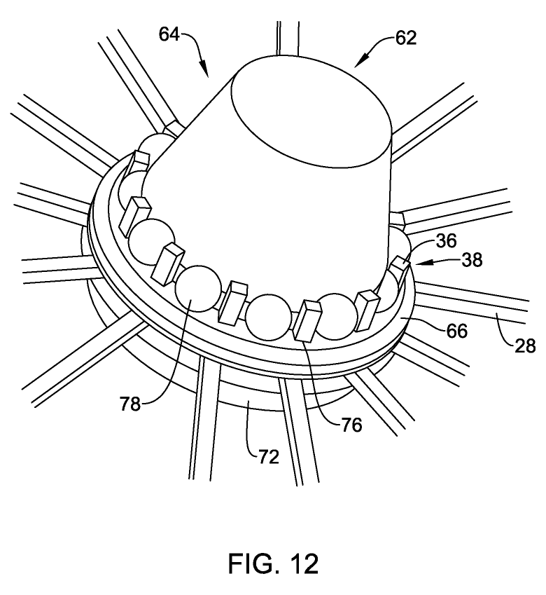

In some cases, the ring member 66 may ultimately be welded to the connection member 64. FIG. 12 is a perspective view showing the connection assembly 62 engaged with the distal tines 28. The ring member 66 is disposed on the connection member 64. A number of individual welds 78 (only one of which are labeled in FIG. 12) secure the ring member 66 to the connection member 64. In some cases, the welds 78 may be the primary means of attaching the ring member 66 to the connection member 64. In some cases, the welds 78 may be used as a secondary means of attaching the ring member 66 to the connection member 64, with the ring member 66 being secured to the connection member 64 via a primary mechanical engagement, a primary snap-fit engagement or a primary threaded engagement, for example.

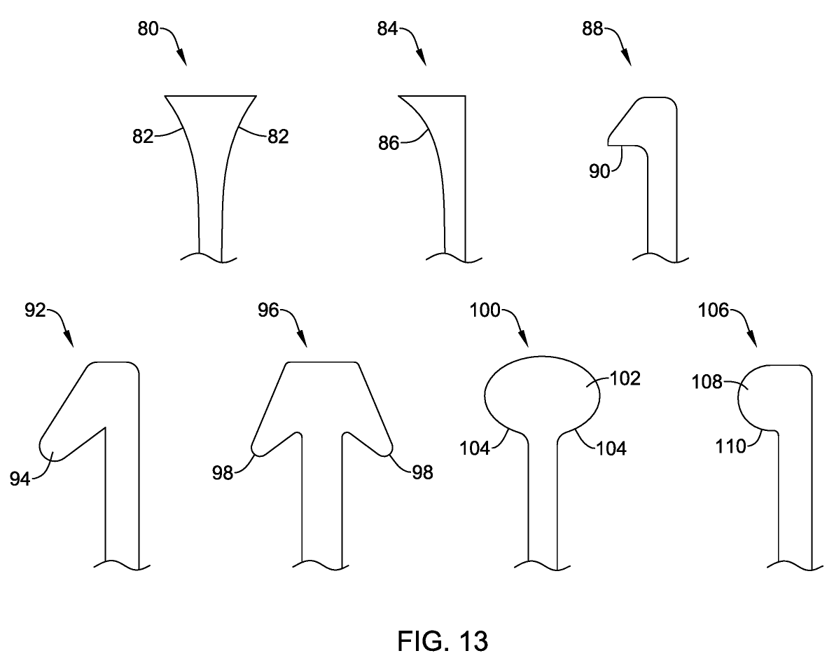

FIG. 13 schematically shows several possible configurations for the engagement portion 38 of each of the distal tines 28. In some cases, the engagement portion 38 of each of the distal tines 28 may have a shape or profile that prevents the engagement portion 38 of each of the distal tines 28 from being pulled out of the corresponding engagement recess 42 within the ring member 34 (or 66). In each case, the profile includes a surface that would engage the ring member 34 (or 66) in response to any attempt to pull the distal tine 28 out of the engagement recess 42. FIG. 13 includes a profile 80 that includes a curved surface 82 on either side that will engage the ring member 34 (or 66). A profile 84 includes a single curved surface 86. A profile 88 includes a flat surface 90 that will engage the ring member 34 (or 66). A profile 92 includes a hook portion 94 that will that will engage the ring member 34 (or 66). A profile 96 includes a hook portion 98 on either side that will engage the ring member 34 (or 66). A profile 100 includes a circular distal region 102 that includes curved surfaces 104 on either side that will engage the ring member 34 (or 66). A profile 106 includes a semi-circular distal region 108 that includes a curved surface 110 that will engage the ring member 34 (or 66).

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Patent Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device, comprising:
an expandable frame extending between a proximal region and a distal region, the distal region including a plurality of tines each having an engagement portion; and
a connection assembly adapted to secure together the engagement portion of each of the plurality of tines, the connection assembly including:
a connection member having an outer surface;
a ring member adapted to be advanced over the outer surface of the connection member, the ring member including an inner annular surface and a plurality of engagement recesses extending from the inner annular surface, each of the plurality of engagement recesses adapted to accommodate the engagement portion of a corresponding one of the plurality of tines;
wherein the engagement portion of each of the plurality of tines is entrapped between the ring member and the connection member, thereby securing the plurality of distal tines.

2. The LAAC device of claim 1, wherein the ring member and the connection member are adapted to provide a threaded engagement therebetween.

3. The LAAC device of claim 1, wherein the ring member and the connection member are adapted to provide a mechanical engagement therebetween.

4. The LAAC device of claim 1, wherein the ring member and the connection member are adapted to provide a snap fit engagement therebetween.

5. The LAAC device of claim 1, wherein the engagement portion of each of the plurality of tines extending between the ring member and the connection member is at least substantially parallel with a longitudinal axis extending through the connection member.

6. The LAAC device of claim 5, wherein a portion of each of the plurality of tines outside of the ring member is at least substantially orthogonal to the longitudinal axis extending through the connection member.

7. The LAAC device of claim 1, wherein the ring member is adapted to be advanced over the connection member after the securement portion of each of the plurality of distal tines are disposed within a corresponding one of the plurality of engagement recesses defined within the inner annular surface of the ring member.

8. The LAAC device of claim 7, wherein the connection member includes a tapered surface in order to allow the ring member to be advanced over the connection member.

9. The LAAC device of claim 1, wherein the connection member further comprises a distal plate that limits relative distal travel of the plurality of distal tines.

10. The LAAC device of claim 8, wherein the connection member comprises one or more axially extending slots that facilitate advancing the ring member over the connection member.

11. A left atrial appendage closure (LAAC) device, comprising:
an expandable frame extending between a proximal region and a distal region, the distal region including a plurality of tines each having an engagement portion; and
a connection member having an outer surface; and
a ring member adapted to be advanced over the outer surface of the connection member in order to entrap the engagement portion of each of the plurality of tines between the ring member and the connection member, the ring member including a plurality of engagement recesses that are adapted to accommodate the engagement portion of a corresponding one of the plurality of tines.

12. The LAAC device of claim 11, wherein the plurality of engagement regions are arranged in order to equally space the engagement portions of each of the plurality of tines.

13. The LAAC device of claim 11, wherein the connection member comprises an annular body portion.

14. The LAAC device of claim 12, wherein the annular body portion comprises a reduced diameter segment that is adapted to accommodate the ring member therein.

15. The LAAC device of claim 13, wherein the connection member comprises a distal segment including a surface that limits distal travel of the plurality of distal tines.

16. The LAAC device of claim 15, wherein the distal segment comprises an atraumatic distal segment.

17. The LAAC device of claim 11, wherein the engagement portion of each of the plurality of tines is adapted to engage a proximal surface of the ring member in order to limit axial travel of the engagement portion relative to the ring member.

18. The LAAC device of claim 11, further comprising an occlusive member extending over at least part of the proximal region of the expandable frame.

19. A left atrial appendage closure (LAAC) device, comprising:
an expandable frame extending between a proximal region and a distal region, the distal region including a plurality of tines each having an engagement portion; and
a connection member; and
a ring member adapted to engage the engagement portion of each of the distal tines prior to advancing the ring member over the connection member to secure the ring member in place relative to the connection member.

20. The LAAC device of claim 19, wherein advancing the ring member over the connection member serves to secure the engagement portion of each of the distal tines between the ring member and the connection member.
